(19) United States
(12) Patent Application Publication
(10) Pub. No.: US 2003/0118662 A1
(43) Pub. Date: Jun. 26, 2003

(54) Therapeutic uses of milk mineral fortified food products

(75) Inventors: Eric Douglas Bastian, Twin Falls, ID (US); Loren Spencer Ward, Twin Falls, ID (US)

Correspondence Address: BANNER & WITCOFF, 1001 G STREET N W, SUITE 1100, WASHINGTON, DC, 20001, US

(73) Assignee: Glanbia Foods, Inc., Twin Falls, ID (US)

(21) Appl. No.: 10/002,011

(22) Filed: Dec. 5, 2001

(51) Int. Cl. A61K 35/20

(52) U.S. Cl. 424/535

(57) ABSTRACT

Food products fortified with a therapeutically effective amount of milk mineral are administered for the treatment of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, or immune deficiency.

### FIELD OF THE INVENTION

The present invention is directed to milk mineral fortified food products and, more particularly to the treatment of high blood pressure, stroke, obesity, and various other disorders by administering food products fortified with a therapeutically effective amount of milk mineral.

### DESCRIPTION OF RELATED ART

The natural milk minerals, especially calcium, magnesium, phosphorus, potassium and zinc, are of great importance in nutrition. Their importance is widely recognized for proper teeth and bone formation, as well as for skeletal structure development. During the period of late teenage to young adulthood, however, significant reductions in dietary calcium intake often occur. This is particularly true of the female population, where reduced dietary calcium intake usually occurs much earlier in life compared to their male counterparts. It has been observed that females are especially susceptible to a prolonged calcium deficit over their life span. This calcium deficit is believed to contribute to the greater incidence of osteoporosis in postmenopausal women.

Calcium supplements and calcium-fortified foods containing calcium in such forms as calcium carbonate, calcium lactate, calcium citrate, calcium chloride, and calcium hydroxide have been proposed. These forms of calcium, however, can yield undesirable flavors and/or can strip desirable aroma and flavor compounds from food products. More significantly, these types of supplements deliver only calcium (no other minerals) and lack the balanced and pure form of the milk minerals (including calcium, phosphorus, potassium, magnesium, and zinc) present in milk and dairy products. As a result, these forms of calcium are less easily absorbed by the body and are inferior to milk and dairy products from a nutritional standpoint.

Some efforts also have been made to isolate milk minerals for use in nutritional supplements. U.S. Pat. No. 5,185,166 to Nakagawa et al., for example, describes a process in which the pH of milk is adjusted to 4-6, after which the whey is ultra-filtered through a membrane. The filtrate is concentrated, and later centrifuged to yield a milk mineral concentrate that is said to contain 25 to 35 percent ash and small amounts of milk minerals, e.g., about 2-10% potassium and about 2-5% calcium. U.S. Pat. No. 5,639,501 to Vembu et al. describes extracting milk minerals from a whey product, such as whey permeate or delactosed whey permeate, using a phosphate compound that is said to keep the milk minerals in solution and in suspension. The purified, dried milk minerals are used in nutritional supplements in the form of tablets, capsules, or liquids.

Whey mineral complexes have been shown to have a more positive effect than calcium carbonate on bone formation. Tsuchita et al., “Comparison of the Effects of Whey Mineral Complexes on Bone Metabolism in Male Growing Rats,” J. Nutr. Sci. Vitaminol 39, 473-487 (1993). While milk mineral supplements have shown promise in the prevention and treatment of osteoporosis, other therapeutic uses of milk mineral-supplemented food products have not been widely explored.

### SUMMARY OF THE INVENTION

The present invention is directed to therapeutic uses of milk mineral fortified foods for the treatment of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, or immune deficiency. According to one aspect of the invention, a method of treating a disorder which is high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, or immune deficiency comprises administering to an individual in need of such treatment a food product containing a therapeutically effective amount of milk mineral. The amount of milk mineral present in the food product can be suitably selected, for example, such that a daily serving or a predetermined number of servings of the food product delivers an amount of milk mineral effective to treat the individual's disorder.

Milk mineral, generally in the form of a powder of appropriate particle size, can be incorporated into a wide variety of types of food products, examples of which include acidic juice beverages (e.g., orange juice, apple juice, grape juice, grapefruit juice, cranberry juice, or blended juices), acidic beverages (e.g., sport beverages, Gatorade®), neutral pH beverages (e.g., milk UHT dairy, RTD nutritional, or soy milk), high-energy protein bars, confectionary products (e.g., high calcium chews, chewing gum, chocolate, or cookies), dairy products (e.g., yogurt, ice cream, milk, cheese, processed cheese, or butter), and bakery products (e.g., bread, biscuits, or rolls). Alternatively, the food product can be a nutritional supplement, such as in the form of tablets or capsules and optionally combined with other minerals and/or vitamins.

According to another aspect of the invention, a method of preparing a milk mineral fortified orange juice product for the treatment of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, or immune deficiency is provided. The method comprises mixing a stream of orange juice with a therapeutically effective amount of milk mineral.

### DETAILED DESCRIPTION OF THE INVENTION

The present invention is directed to methods of treating a variety of disorders by administering a food product containing a therapeutically effective amount of milk mineral. A first group of disorders including high blood pressure, stroke, obesity, kidney stones, colon cancer, breast cancer, head and neck tumors, premenstrual syndrome, postpartum depression, and hypertensive disorders of pregnancy, have been associated to some degree with inadequate intake of dairy products, and more particularly the minerals present in dairy products. A second group of disorders including Type-2 diabetes, depression, asthma, inflammatory bowel disease, attention deficit disorder, migraine headaches, kidney disease, hypercholesterolaemia, congestive heart failure, and immune deficiency have been clinically associated with the first group of disorders and exhibit similar pathophysiologic mechanisms. It has been discovered that both the first group and the second group of disorders can be effectively treated by administering food products fortified with a therapeutically effective amount of milk mineral in accordance with the practice of the present invention.

The terms “treat,” “treating,” “treatment,” and similar terms as used herein refer to the administration of the fortified food products to individuals, particularly humans, suffering from one or more of the specified disorders, for alleviating, suppressing, inhibiting, or otherwise reducing the extent of the disorder(s) or any symptom thereof. The terms “treat,” “treating,” “treatment,” and similar terms also are used herein to refer to the prophylactic administration of the fortified food products to individuals who may be at risk of developing one or more of the disorders.

The term “milk mineral,” as used herein, refers to a mineral complex obtained from whey or milk. The mineral complex contains a balanced form of calcium, phosphorus, potassium, magnesium, and zinc. Milk mineral has a relatively neutral taste, in contrast to the chalky taste of calcium carbonate. Whey fractions that are high in calcium have been demonstrated to exhibit higher calcium bioavailability than are exhibited by calcium carbonate and calcium lactate. Ranhotra et al., “Bioavailability of Calcium in a High Calcium Whey Fraction,” Nutrition Research, Vol. 17 Nos. 11-12, pp. 1663-1670 (1997). For optimal absorption, calcium and phosphorous preferably are present in a calcium-to-phosphorous ratio of about 1:1 to 2:1, e.g., a ratio similar to that found in both milk and in bone. The milk mineral also typically contains quantities of lactose and bioactive proteins. Whey proteins occur in milk as soluble, globular proteins. The primary proteins and peptide constituents derived therefrom are α-lactalbumin and β-lactoglobulin, kappa-casein fragment 106-109 (caseino-glycomacropeptide), lactoferrin, bovine serum albumin, lactoperoxidase, and immunoglobulins. Milk mineral is also commonly referred to as “milk calcium.”

Suitable methods of obtaining milk mineral by extraction from whey or milk are known to persons skilled in the art. One suitable extraction method is described in U.S. Pat. No. 5,639,501, the disclosure of which hereby is incorporated by reference in its entirety. A typical composition of milk mineral is illustrated in Table 1 below.

TABLE 1
Typical Composition for Milk mineral Powder

| Component | Relative Amount (% by weight) |
|---|---|
| Total Minerals | 60-90% |
| Inorganic Mineral (Ash) | 50-85% |
| Organic Mineral (Citrate) | 1-10% |
| Calcium | 15-30% |
| Phosphorous | 7-17% |
| Lactose | 0-15% |
| Protein | 5-15% |
| Free Moisture | 2-5% |
| Fat | 0-5% |

The milk mineral extract typically is purified, spray dried, and ground into a powder having an appropriate particle size to permit mixing into a desired liquid or solid food product. Suitable particle sizes will depend on such factors as the physical properties (e.g., liquid or solid, specific gravity, pH, viscosity, etc.) of the food product into which the powder is mixed. The mean particle size most often ranges from about 0.1 microns to about 300 microns, more usually from about 1 micron to about 100 microns. For neutral pH beverages, such as milk, a more finely ground powder preferably is employed so that a suspension of the powder can be easily formed. Because the solubility of the powder increases as pH decreases, less finely ground powders typically can be used, for example, in acidic juice beverages and in acidic beverages, in which the milk mineral powder solubilizes.

The milk mineral powder can be mixed together with a wide variety of types of food products, including acidic juice beverages (e.g., orange juice, apple juice, grape juice, grapefruit juice, cranberry juice, or blended juices), acidic beverages (e.g., sport beverages, Gatorade®), neutral pH beverages (e.g., milk UHT dairy, RTD nutritional, or soy milk), high-energy protein bars, confectionary products (e.g., high calcium chews, chewing gum, chocolate, or cookies), dairy products (e.g., yogurt, ice cream, milk, cheese, processed cheese, or butter), and bakery products (e.g., bread, biscuits, or rolls). Alternatively, the food product can be a nutritional supplement, such as in the form of tablets or capsules, optionally combined with other minerals and/or vitamins, in accordance with techniques well known to persons skilled in the art.

The relative amount by weight of milk mineral combined with the food product depends on such factors as the density and the serving size of the food product. Typically, the amount of milk mineral ranges from 0.1 to about 10 percent by weight, based on the total weight of the food product. The amount of milk mineral added to milk may be selected, for example, to provide about 400-450 mg of calcium in an 8 oz. serving. Fortified milk can be formulated to contain lactose or can be formulated to be free of lactose. In either case, the fortified milk optionally can be formulated to have reduced (or zero) milk fat.

One important attribute of milk mineral is the calcium-to-magnesium ratio. Improper calcium-to-magnesium ratios have been shown to lead to leaching of other important minerals, which in turn may lead to bone brittleness and can even increase the risk of osteoporosis. High dietary Ca:Mg ratios interfere with magnesium absorption because calcium and magnesium share common intestinal absorption pathways. When calcium levels are high with respect to magnesium levels, calcium competes with magnesium for the absorption pathways, resulting in hypomagnesaemia (low magnesium in the blood).

Milk mineral contains a balance of minerals and bioactive proteins, which renders the fortified food products of the present invention effective in the treatment of a variety of disorders including high blood pressure, stroke, obesity, kidney stones, colon cancer, breast cancer, head and neck tumors, premenstrual syndrome, postpartum depression, and hypertensive disorders of pregnancy. Another group of disorders including Type-2 diabetes, depression, asthma, inflammatory bowel disease, attention deficit disorder, migraine headaches, kidney disease, hypercholesterolaemia, congestive heart failure, exhibit similar pathophysiologic mechanisms and also can be effectively treated with the milk mineral fortified food products of the present invention. While not wanting to be bound by theory, the following provides a discussion of the mechanisms by which the various types of disorders are treated.

Calcium is essential for muscle function regulation, blood clotting, hormone regulation, nerve function, and enzyme activation. Calcium in milk mineral has a high bioavailability, which is enhanced by vitamin D, lactose, gastrointestinal acidity, and certain fibers. Also, the balanced form of calcium, phosphorous, potassium, magnesium, zinc, and vitamin D in milk mineral helps to minimize calcium depletion through urinary loss. The calcium present in the milk mineral supplemented food product provides high calcium bioavailability effective for the treatment of colon cancer, and head and neck tumors. The high calcium bioavailability of the milk mineral fortified food products also is effective for the treatment of obesity, hypertension (including hypertension-related disorders such as high blood pressure), gestational hypertension, hypochloestemia, premenstrual syndrome, postpartum depression, and breast cancer.

### EXAMPLES

The following examples are given for purposes of illustration and should not be regarded as limiting the scope of the invention as set forth in the appended claims.

### Example 1

This example illustrates preparing a lemon-lime beverage fortified with milk mineral. The milk mineral powder was ground such that 90% of the particles had a particle size of less than 50 micron (bulk density 0.89 g/ml). The components were mixed to yield a product containing:

| Ingredient | Amount (wt %) |
|---|---|
| Water | 90 |
| Sugar | 8.9 |
| Milk mineral | 0.52 |
| Flavor | 0.1 |
| Colors: yellow #5, blue #1 | 0.0001 |
| Citric acid | 0.25 |
| 75% phosphoric acid | 0.54 |

An 8 oz. serving of the fortified beverage provides 300 mg of calcium (30% RDI), equivalent to an 8 oz. serving of milk.

### Example 2

This example illustrates preparing fortified chocolate chip cookies. The milk mineral powder was ground such that 90% of the particles had a particle size of less than 7 micron (bulk density 0.59 g/ml). The following components were combined in a bowl and mixed minimally to blend.

| Component | Amount (wt %) |
|---|---|
| Unbleached flour | 24 |
| Fat + sugar blend | 34.8 |
| Corn Syrup | 13 |
| Salt | 0.2 |
| Baking soda | 0.7 |
| Milk mineral | 6.5 |
| Water | 2.2 |
| Chocolate flavor | 0.15 |
| Caramel color | 0.3 |
| Chocolate chips | 18.2 |

The dough was scooped into 11 g portions, applied to an ungreased pan, and baked at 350° F. for 10 minutes. Water was added to offset water-binding of the milk mineral, which can result in cookies with less spread. The cookies contained 500 mg of calcium per serving and had a good taste.

### Example 3

This example illustrates preparing milk mineral-fortified bread using the powdered milk mineral described in Example 1 or Example 2. The following components were used:

| Component | Amount (Baker's percent) |
|---|---|
| SPONGE | |
| flour | 80.0 |
| instant yeast | 1.3 |
| water | 47.62 |
| DOUGH | |
| flour | 20.0 |
| instant yeast | 10.7 |
| salt | 2.0 |
| Dimodan ® ESK | 0.375 |
| Panodan ® 205 | 0.25 |
| HFCS | 12.0 |
| Vegetable oil | 3.0 |
| Ascorbic acid (1:99 aqueous solution) | 60 (ppm) |
| Water | 10.0 |
| Milk mineral | 3.9 |

The sponge was mixed for 1 minute on low and for 3 minutes on medium. The sponge was fermented for 3 hours at 90° F. and 85% relative humidity. The sponge then was added to the dough and mixed for 1 minute on low and for 8 minutes on medium. After 5 minutes, the dough was placed into a pan and proofed for 60 minutes at 110° F. and 85% relative humidity, and thereafter baked at 425° F. for 22 minutes. The fortified bread contains 150 mg calcium per ounce, and has a specific bread volume of 6.9 cc/g (when using the milk mineral powder of Example 1) or 7.1 cc/g (when using the milk mineral powder of Example 2).

### Example 4

This example illustrates preparing a fortified mint calcium chew. A frappe was preparing by combining HFCS or invert sugar (49.5 wt %), dried egg albumin (1.0 wt %), and 42DE corn syrup (49.5 wt %). A syrup base was prepared by combining 42DE corn syrup (41.7 wt %), sugar (34.1 wt %), water (14.9 wt %), hydrogenated palm kernel oil (8.5 wt %), glycerol monostearate (0.5 wt %), and 150 bloom gelatin (0.3 wt %), and heating to 200° F. The frappe was thoroughly mixed into the syrup base, and milk mineral (20 wt % based on total weight of candy chew) was mixed in at low speed. The mass was blended at high speed for 3 minutes. Peppermint oil (0.2 wt %) was then added and the mass mixed at low speed for one minute. The product thereafter was removed from the beater and cooled on a cooling table or drum until its texture was suitable for extrusion or rolling. A package of 3 chews contains 500 mg of calcium.

### Example 5

This example illustrates preparing milk mineral-fortified orange juice. To a stream of orange juice was added 23 g/gal. of the powdered milk mineral as described in Example 1. Citric acid solids (0-7.5 g/gal.) may be added. The fortified orange juice product contains 350 mg of calcium in an 8 oz. serving.

### Example 6

This example illustrates preparing a yogurt drink fortified with milk mineral. A fruit concentrate (69.6 wt %) and high-methoxy pectin (0.4 wt %) were heated to 90° C. under shear, and then cooled to 50° C. Yogurt (29.8%) was added together with milk mineral (0.12 wt %) and homogenized under pressure at 40-45° C. The mass was heated to 90° C. for 15 sec., and then was bottled at 20° C. and stored at 7° C. for 7 days. The resulting drink exhibited stability and viscosity similar to a non-fortified drink. It was found that milk mineral could be added either to the fruit concentrate during syrup production or to the final yogurt blend before processing. The pH of the drink during production is sufficient to dissolve the milk mineral particles, so either the powder of Example 1 or that of Example 2 can be used. The fortification results in a 50% increase in the calcium content of the yogurt drink.

While particular embodiments of the present invention have been described and illustrated, it should be understood that the invention is not limited thereto since modifications may be made by persons skilled in the art. The present application contemplates any and all modifications that fall within the spirit and scope of the underlying invention disclosed and claimed herein.

What is claimed is:

1. A method of treating a disorder selected from the group consisting of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, and immune deficiency, the method comprising administering to an individual in need of such treatment a food product containing a therapeutically effective amount of milk mineral.

2. The method of claim 1, wherein said milk mineral comprises:

| | |
|---|---|
| Total Minerals | 60-90% |
| Inorganic Mineral | 50-85% |
| Organic Mineral | 1-10% |
| Calcium | 15-30% |
| Phosphorous | 7-17% |
| Lactose | 0-15% |
| Protein | 5-15% |
| Fat | 0-5% |

by weight, based on the total weight of said milk mineral.

3. The method of claim 1, wherein said disorder is selected from the group consisting of high blood pressure, stroke, obesity, kidney stones, colon cancer, breast cancer, head and neck tumors, premenstrual syndrome, postpartum depression, and hypertensive disorders of pregnancy.

4. The method of claim 1, wherein said food product is selected from the group consisting of acidic juice beverages, acidic beverages, neutral pH beverages, high energy protein bars, confectionary products, dairy products, and bakery products.

5. The method of claim 4, wherein said food product comprises an acidic juice beverage.

6. The method of claim 5, wherein said food product comprises orange juice.

7. The method of claim 1, wherein said food product comprises from about 0.1 to about 10 wt % of said milk mineral, based on the total weight of said food product.

8. A method of preparing a milk mineral fortified orange juice product for treating a disorder selected from the group consisting of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, and immune deficiency, the method comprising mixing a stream of orange juice with a therapeutically effective amount of milk mineral.

9. The method of claim 8, wherein said milk mineral comprises:

| | |
|---|---|
| Total Minerals | 60-90% |
| Inorganic Mineral | 50-85% |
| Organic Mineral | 1-10% |
| Calcium | 15-30% |
| Phosphorous | 7-17% |
| Lactose | 0-15% |
| Protein | 5-15% |
| Fat | 0-5% |

by weight, based on the total weight of said milk mineral.

10. The method of claim 8, wherein said disorder is selected from the group consisting of high blood pressure, stroke, obesity, kidney stones, colon cancer, breast cancer, head and neck tumors, premenstrual syndrome, postpartum depression, and hypertensive disorders of pregnancy.

11. The method of claim 8, wherein said orange juice comprises from about 0.1 to about 10 wt % of said milk mineral, based on the total weight of said fortified orange juice product.

12. A milk mineral fortified orange juice product for treating a disorder selected from the group consisting of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolaemia, congestive heart failure, and immune deficiency, comprising orange juice and therapeutically effective amount of milk mineral.

13. The milk mineral fortified orange juice product of claim 12, wherein said milk mineral comprises:

| | |
|---|---|
| Total Minerals | 60-90% |
| Inorganic Mineral | 50-85% |
| Organic Mineral | 1-10% |
| Calcium | 15-30% |
| Phosphorous | 7-17% |
| Lactose | 0-15% |
| Protein | 5-15% |
| Fat | 0-5% |

by weight, based on the total weight of said milk mineral.

14. The milk mineral fortified orange juice product of claim 12, wherein said disorder is selected from the group consisting of high blood pressure, stroke, obesity, kidney stones, colon cancer, breast cancer, head and neck tumors, premenstrual syndrome, postpartum depression, and hypertensive disorders of pregnancy.

15. The milk mineral fortified orange juice product of claim 12, wherein said orange juice comprises from about 0.1 to about 10 wt % of said milk mineral, based on the total weight of said fortified orange juice product.
